(19) United States
(12) Patent Application Publication
(10) Pub. No.: US 2009/0053149 A1
(43) Pub. Date: Feb. 26, 2009

(54) SURFACTANT-BASED ANTIMICROBIAL SOLUTION FOR INHALATION

(75) Inventors: Tim Corcoran, Pittsburgh, PA (US); Amy Lise Marcinkowski, Syracuse, NY (US); Joseph Pilewski, Pittsburgh, PA (US); Kristina Thomas, Pittsburgh, PA (US)

Correspondence Address: FOLEY AND LARDNER LLP;SUITE 500, 3000 K STREET NW, WASHINGTON, DC, 20007, US

(73) Assignee: University of Pittsburgh-Of the Commonwealth System of Higher Education

(21) Appl. No.: 12/195,544

(22) Filed: Aug. 21, 2008

Related U.S. Application Data
(60) Provisional application No. 60/957,925, filed on Aug. 24, 2007.

(51) Int. Cl. A61K 9/12; A61K 31/7036; A61K 31/546; A61K 31/427; A61K 38/12; A61K 31/7052; A61K 31/155; A61K 31/65; A61P 31/10; A61P 31/04; A61P 31/12; A61K 31/5377; A61K 31/407; A61K 31/44; A61K 31/496; A61K 31/351; A61K 31/7048

(52) U.S. Cl. 424/45; 514/40; 514/203; 514/210.15; 514/10; 514/29; 514/636; 514/152; 514/236.8; 514/210.13; 514/354; 514/254.11; 514/459; 514/31

(57) ABSTRACT

A surfactant can be added, safely and effectively, to a drug solution containing any antimicrobial agent, such as an antibiotic like tobramycin, that is suitable for administration to the lungs via inhalation. Thus, when an aerosolized drug solution includes surfactant, Marangoni flows cause the drug particles, once deposited in the lungs, to spread over a wider surface area, thereby ensuring greater antimicrobial efficacy. A solution that contains, for example, an antibiotic and tyloxapol or another surfactant providing a similar surface tension to the composition is optimally delivered by the functional combination of a breath-actuated nebulizer and a high-flow compressor.

### RELATED APPLICATIONS

This application claims priority from U.S. Provisional Application No. 60/957,925, filed Aug. 24, 2007, which is incorporated herein by reference in its entirety.

### BACKGROUND OF THE INVENTION

The present invention relates to a pharmaceutical composition for treating microbial infections in the body, specifically in the lung, and to a system for aerosol administration of the composition.

Inhalation therapies were put forward in the mid-1900s, when nebulized asthma drugs became available, but such therapies were only poorly realized. The late 1990s saw the development of an inhalable vehicle for insulin, an approach that has proven as reliable as insulin injection. See “Inhaling medicines: Delivering drugs to the body through the lungs,” Nature Rev. Drug Discov. 6: 67-74 (2007) (hereafter, “2007 review article”).

Aerosolized medications for cystic fibrosis patients, who suffer numerous recurring lung infections, have played a role in conventional antimicrobial regimens. The latter have been unreliable, however, because pulmonary infections are difficult to target in the complex branching that characterizes the internal structure of the human lung.

Novartis has developed an aerosolized treatment that employs “Tobramycin Solution for Inhalation” (TOBI), which has been shown to improve patient outcomes when added to the standard regimen of medications used to treat cystic fibrosis. TOBI is a saline-based solution of tobramycin, an antibiotic, which was first used against P. aeruginosa in the 1970s and then adapted to inhalation in the mid-to-late 1990s.

The TOBI regimen offers the advantages of (1) a specific dosage that studies have verified has an acceptable level of safety and effectiveness and (2) a recommended delivery system that provides adequate deposition of the drug solution to target sites in the lungs. Although the TOBI regimen can achieve bacterial suppression, it does not eradicate infection fully. Over the course of clinical trials, for example, TOBI reduced bacterial density during administration but did not prevent a return of bacterial density to baseline levels, post-administration. Apparently failing to reach all bacterial reservoirs in the lungs, in other words, TOBI's aerosol particles did not eradicate the source of the infection.

### SUMMARY OF THE INVENTION

To address this issue and to achieve other practical advantages for inhaled medicaments, the present invention provides, in accordance with one aspect, a sterile, isotonic aqueous composition comprised of (i) an antimicrobial agent and (ii) a non-ionic surfactant in an amount such that nebulization of the composition yields an aerosol characterized by a median droplet size in the range of about 1 to 5 μm, which composition does not comprise phospholipids. Preferably, the composition has a surface tension of about 35 dynes/cm and, more preferably, of less than about 35 dynes (mN) per centimeter (cm). The primary determinant of surface tension in this context is the surfactant, which preferably is tyloxapol, present in an amount that is less than about 1% by mass, e.g., about 0.1% by mass.

The antimicrobial agent may be an antibiotic, an antifungal, or an antiviral agent, or a combination of any of these. Illustrative of suitable antimicrobial agents are: (A) tobramycin, amakacin, ceftazidime, aztreonam, colistin, ciprofloxacin, azithromycin, pentamidine, and gentamicin; (B) vancomycin, doxycycline, linezolid, meropenem, and tigecycline; (C) isoniazid, rifampin, and daptomycin; (D) Amphotericin B; and (E) zanamivir and oseltamivir. In a preferred embodiment, the antimicrobial agent is tobramycin, present in an amount up to about 10% by mass.

In accordance with another aspect of the invention, a combination is provided for delivering an antimicrobial agent to the lungs by oral inhalation, comprising (A) a breath-actuated nebulizer operatively connected to (B) a high-flow compressor that delivers to the nebulizer a gas flow greater than 5 L/min and a pressure head of at least 40 psi, where the nebulizer contains a liquid to be atomized that is an aqueous composition as described above. Pursuant to a further aspect, the invention provides a method for delivering an antimicrobial agent to the lungs, comprising (A) forming an aerosol of such an aqueous composition, where said aerosol is characterized by a median droplet size in the range of about 1 to 5 μm, and (B) delivering that aerosol to a subject for inhalation, such that said subject receives aerosol only during inhalation.

### BRIEF DESCRIPTION OF THE DRAWINGS

FIGS. 1A and 1B provide a series of graphs that display the results of studies demonstrating a proportional relationship between tobramycin mass and radioactive counts when an SBTSI-Technetium DTPA solution was nebulized.

FIG. 2 is a histogram that compares aerosol volume distribution for a SBTSI solution versus a tyloxapol-only solution of the same surfactant concentration.

FIG. 3 is a graph that displays predicted total and pulmonary deposited tobramycin for each of the tested eleven delivery systems.

FIG. 4 is a graphical depiction of the pulmonary delivery rate for each of eleven delivery systems.

FIG. 5 is graph that shows the predicted delivery rate of medication to the large bronchial airways of the lungs.

FIG. 6 is a graph depicting the predicted aerosol volume distribution by lung region for SBTSI used with a preferred nebulizer-compressor combination.

### DETAILED DESCRIPTION OF THE PREFERRED EMBODIMENTS

In accordance with the present invention, the use of a surfactant in an inhalation therapy causes the active agent or drug to disperse over a greater surface area of the target site, after aerosol deposition. As the present inventors have discovered, when an aerosolized drug solution includes a significant surfactant concentration, then Marangoni flows cause the drug particles, once deposited on the target site, to spread over a wider surface area.

In other contexts, surfactants have displayed an effective dispersing ability and have a good safety record for in vivo use, particularly in the lungs. To the inventors' knowledge, however, no FDA-approved inhalation therapy has ever utilized surfactants to provide wider dispersion of an inhaled medication over a target site in the lungs. See the 2007 review article, supra, for example. Indeed, no conventional inhalation therapy has utilized any substance to provide wider deposition of the aerosol particles, once they deposit on a target site in the lungs.

The addition of a surfactant, pursuant to the present invention, aids the dispersion of aerosol particles over a target site for an inhalation therapy that employs any antimicrobial agent suitable for administration to the lungs. To this end, a pharmaceutical composition of the invention contains surfactant such that the surface tension of the composition, measured in conventional fashion, is about 35 mN/cm or less, the numerical value being approximately the surface tension of the endogenous liquid that lines the large airways of the human lung. See, e.g., I m Hof et al., Respir. Physiol. 109: 81-93 (1997), and Tarran et al., J. Gen. Physiol. 118: 223-36 (2001). The lower end of the surface-tension range for the present invention is the lowest value measured heretofore in surfactant solutions delivered to the lungs for surfactant replacement therapy, which is on the order of 3 to 4 mN/cm.

An actual determination of surface tension typically is not a prerequisite to preparing a composition of the present invention. Rather, pre-clinical development of an antimicrobial formulation for pulmonary delivery, in accordance with the invention, generally will proceed along empirical lines, and the amount of surfactant employed thus will be adjusted to accommodate the salient parameters of the given application. Accordingly, the foregoing “about 35 mN/cm or less” prescription is meant to convey not a precise cutoff but rather the general guidance that surface tension, in the given circumstance, should approximate or, more preferably, should be lower than the endogenous liquid layer that conditions the pulmonary target site(s).

Where it proves desirable, in any event, surface tension of a composition can be measured via any suitable technique, of which a number are conventionally available. For example, see PHYSICAL CHEMISTRY OF SURFACES, 6^{th} ed. (John Wiley & Sons, Inc.), pages 16-33. One such technique, the so-called “ring method,” employs the de Noüy ring system, in which a metallic ring of platinum and/or iridium is applied to measure surface tension. Id., pages 21-23. The ring forms an interface with the test liquid, and a tensiometer measures the tensile strength required to detach the ring from the liquid surface.

The de Noüy ring system is versatile and suitable for approximating the surface tension of a composition with the present invention. By way of illustration, the formulation of Example 1, comprised of tobramycin (antibiotic) and tyloxapol (surfactant), was measured three time with a de Noüy ring system, yielding values of 36.2, 37.2, and 37.2 dynes/cm. Accordingly, this method provided an average surface tension of 36.6±0.47 dynes/cm, which, for the applicable circumstances, was “about” 35 dyes/cm and operationally effective. By the same token, surface tension may be measured, as such, to inform pre-clinical development, pursuant to this invention, of a formulation that has a surface tension value that is “about” 35 dynes/cm or lower.

The chosen surfactant should not interfere with the medicinal properties of an administered composition that derives from the antimicrobial agent so employed. The surfactant preferably is non-ionic; that is, it is uncharged and not prone to dissociate in water.

The employed antimicrobial agent includes but is not limited to a number of antibiotic, antifungal, and antiviral agents, as discussed in detail below. To ensure that it is appropriate for clinical applications, a pharmaceutical composition of the invention should be aqueous and sterile, and should have essentially the same concentration of solutes as human blood, i.e., should be “isotonic.”

Moreover, a composition of the present invention preferably does not contain phospholipids in any amount. More particularly, the invention preferably excludes the presence of colloidal structures such as liposomes, micelles, anisotropic liquid crystals, liquid crystalline mesophases, and complexes that include phospholipids or that are the direct result of the addition of phospholipids. Compare U.S. published patent application No. 2005/0244339.

Likewise in support of adequate dispersion of an inhaled composition of the invention, the aerosol particles should be the correct size to deposit in the targeted region(s) of infection. For effective treatment in the lungs, therefore, the median aerosol particle size for the present invention is preferably in the range of 1 to 5 μm.

The aerosolization characteristics of a pharmaceutical composition within the invention is determined primarily by the surfactant. With this understanding, the inventors have discovered that a certain delivery system is surprisingly efficacious for delivering to the lungs a pharmaceutical composition solution of the invention that contains the surfactant tyloxapol (“tyloxapol-based pharmaceutical composition”). The inventors tested numerous nebulizer/compressor combinations and used an in vitro model, as described below, to determine that the best device combination for tyloxapol-based pharmaceutical compositions, pursuant to the invention, is a breath-actuated nebulizer in a functional connection with a high-flow compressor. This delivery system also should be efficacious when tyloxapol is replaced by another surfactant that induces a similar solution surface tension (see discussion below).

An “antimicrobial agent” in this context can be an antibiotic agent, an antiviral agent, or an antifungal agent, depending on the source of infection in the body. The following enumeration of exemplary antimicrobial agents is not exclusive of those that are suitable for the present invention. Similarly illustrative are the microorganisms mentioned that can be targeted by a surfactant-based antimicrobial solution for inhalation, pursuant to the invention.

An antibiotic agent treats gram negative bacteria, including Pseudomonas aeruginosa, and gram positive bacteria, including Streptococcus penumoniae and Staphylococcs aureus. Tobramycin is FDA-approved as an aerosolized antibiotic treatment for Pseudomonas aeruginosa. Other antibiotic drugs that have some development in an aerosolized form include amakacin, ceftazidime, aztreonam, colistin, ciprofloxacin, azithromycin, pentamidine, and gentamicin. Vancomycin, doxycycline, linezolid, meropenem, and tigecycline are antibiotic drugs that might also be successfully used in an inhaled form. Isoniazid, rifampin, and daptomycin should be utilized if the source of the infection is tuberculosis or other mycobacteria.

An antifungal agent targets a fungus such as Aspergillius fungi. Amphotericin B or other appropriate antifungal agents should be used in the administered drug solution if the source of the infection is a fungus.

An antiviral agent acts against a virus such as influenza. Thus, the present invention contemplates the use, in the manner described above, of zanamivir, oseltamivir, or any other antiviral agent that is appropriate for administration to the lungs.

As a function of the antimicrobial agent employed, the amount of agent in a pharmaceutical composition of the invention will vary, e.g., as a percentage by mass. The tobramycin content in TOBI, for instance, is approximately 6% by mass, or 300 mg of tobramycin for each 5 ml of drug solution. For the invention, by the same token, the tobramycin content of a drug solution can be up to about 10% by mass.

A formulation of the present invention may include any non-ionic surfactant that safely and effectively lowers the surface tension of an aerosolized drug solution, in keeping with the description above. The amount of surfactant as a percentage by mass will vary among aerosolized drug solutions administered pursuant to the invention. For instance, when the surfactant in the drug solution is tyloxapol, the surfactant content should be between 0.01 and 1% by mass, or between about 0.5 and 50 mg of tyloxapol for each 5 ml of drug solution.

As noted, the inventors discovered the optimal delivery system for a pharmaceutical composition of the invention in which the surfactant is tyloxapol or another compound that affords, to the drug solution, a surface tension and, preferably, a viscosity and a density similar to the corresponding values afforded by tyloxapol. Thus, a formulation of the invention could include any one or more non-ionic surfactant selected from the group of: polysorbate 20, 40, 60, 65, 80, 81 and 85; sorbitan monopalmitate; sorbitan monostearate; sorbitan tristearate; sorbitan monooleate; and sorbitan trioleate. The dispersion characteristics of such a formulation can be verified via the in vitro methodology described in Example 2 below.

The optimal delivery system of the invention utilizes (i) a nebulizer that creates an aerosol only when the patient inhales (“breath-actuated nebulizer”), which maximizes the amount and rate of drug deposition in the body, with (ii) a “high-flow compressor,” which is a compressor that delivers gas at a rate greater than five liters per minute. The high-flow compressor also delivers a pressure head of 40 psi to the breath-actuated nebulizer. In a preferred embodiment, the breath-actuated nebulizer/high-flow compressor combination of the present invention comprises an AeroEclipse II Breath-Actuated Nebulizer in functional connection with a DeVilbiss 8650D high-flow compressor.

A delivery system of the invention also includes, typically integral with the nebulizer, (iii) a reservoir for storing a drug solution to be atomized by the nebulizer, and (iv) a conduit adapted for delivery of the aerosol to the user. The reservoir can be a cartridge, tube, cup, or any container that stores fluid. The conduit can be a hose, pipe, connector, tube, or any transport device that may be used to transport gas to a face mask, nozzle or any other device that delivers gas to the respiratory system.

This delivery system yields an aerosol with a median aerosol particle size that is effective for deposition of the drug solution on the target site in the lungs, especially the lung airways. For example, the delivery device combination can yield a median aerosol particle size between 1 and 5 μm, which is the particle size that is most effective for depositing in the lung branches.

The present invention is further described by reference to the following examples, which are illustrative only and not limiting of the claimed invention.

### EXAMPLE 1

### Surfactant-Based Composition and Delivery Device

The inventors produced a surfactant-based tobramycin solution for inhalation (“SBTSI”). Each 10 ml of SBTSI comprised 0.5 ml of 20 mg/ml tyloxapol solution, 600 mg of tobramycin, and 43 mg of NaCl, with added water to reach 10 ml. The solution did not include phospholipids.

In testing a delivery system for SBTSI, the inventors considered eleven aerosol delivery systems, each including a nebulizer and a compression source (see Table 1). The inventors employed a solution containing only the tyloxapol component of SBTSI, essentially to save the cost of repeated uses of the antibiotic, tobramycin. For testing purposes, this expediency was acceptable in principle because the surfactant component was the dominant factor affecting aerosolization.

TABLE 1
Nebulizer delivery systems included in study
to determine optimal system for SBTSI.

| System | Nebulizer | Manufacturer | Compressor | Manufacturer |
|---|---|---|---|---|
| 1 | Acorn II | Vital Signs Inc. | 8650D | DeVilbiss |
| 2 | AeroEclipse | Monaghan | 8650D | DeVilbiss |
| | II BAN | Medical | | |
| 3 | AeroEclipse | Monaghan | PulmoAide | DeVilbiss |
| | II BAN | Medical | | |
| 4 | Aerotech II | CIS-US Inc. | 8650D | DeVilbiss |
| 5 | Aerotech II | CIS-US Inc. | PulmoAide | DeVilbiss |
| 6 | Micromist | Hudson RCI- | 8650D | DeVilbiss |
| | | Teleflex | | |
| 7 | Pari Star | Pari Inc. | 8650D | DeVilbiss |
| 8 | Updraft II | Hudson RCI- | 8650D | DeVilbiss |
| | | Teleflex | | |
| 9 | Updraft II | Hudson RCI- | PulmoAide | DeVilbiss |
| | | Teleflex | | |
| 10 | Updraft II w/ | Hudson RCI- | PulmoAide | DeVilbiss |
| | Medicator | Teleflex/Health- | | |
| | Resevoir* | line Med. | | |
| 11 | Pari Plus | Pari Inc. | 8650D | DeVilbiss |

| Manufacturer | Location | Website |
|---|---|---|
| CIS-US Inc. | Bedford, MA | www.cisusinc.com |
| DeVilbiss | Somerset, PA | www.sunrisemedical.com |
| Healthline Medical | Baldwin Park, CA | www.healthlinemed.com |
| Hudson-RCI | Research Triangle | www.teleflexmedical.com |
| | Park, NC | |
| Monaghan Medical | Plattsburgh, NY | www.monaghanmed.com |
| Pari Inc. | Midlothian, VA | www.pari.com |

*Included the use of Medicator Aerosol Maximizer aerosol resevoir adjunct which collects aerosol normally lost during patient exhalation making it availing during the next patient inhalation.

The inventors also added a Technetium DTPA radioisotope tag to the tyloxapol solution. The radioactivity associated with the Technetium DTPA allows for accurate measurement of liquid volume output when the aerosol is collected in collection filters. That is, the counts of radioactivity in a filter informs one of the percentage of volume output. Knowledge of the drug content per unit volume with the SBSTI solution then permits one to estimate drug output does. Thus, liquid volume output of the tyloxapol solution is proportional to tobramycin output of the SBSTI solution (see below).

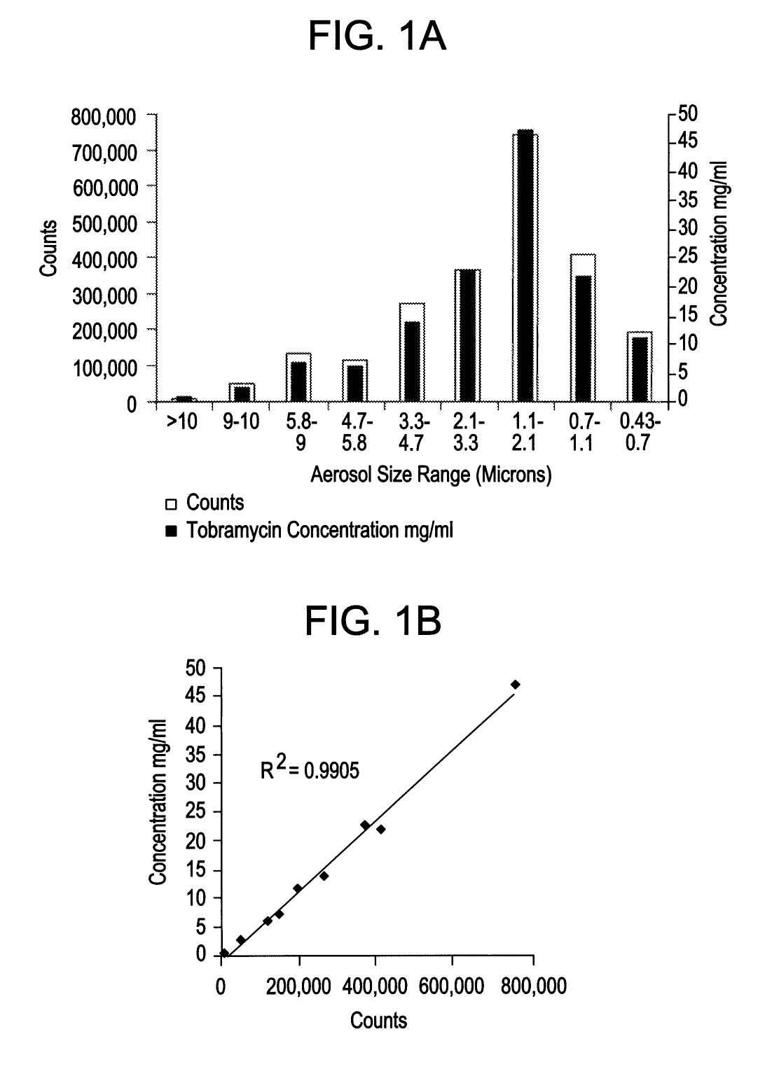

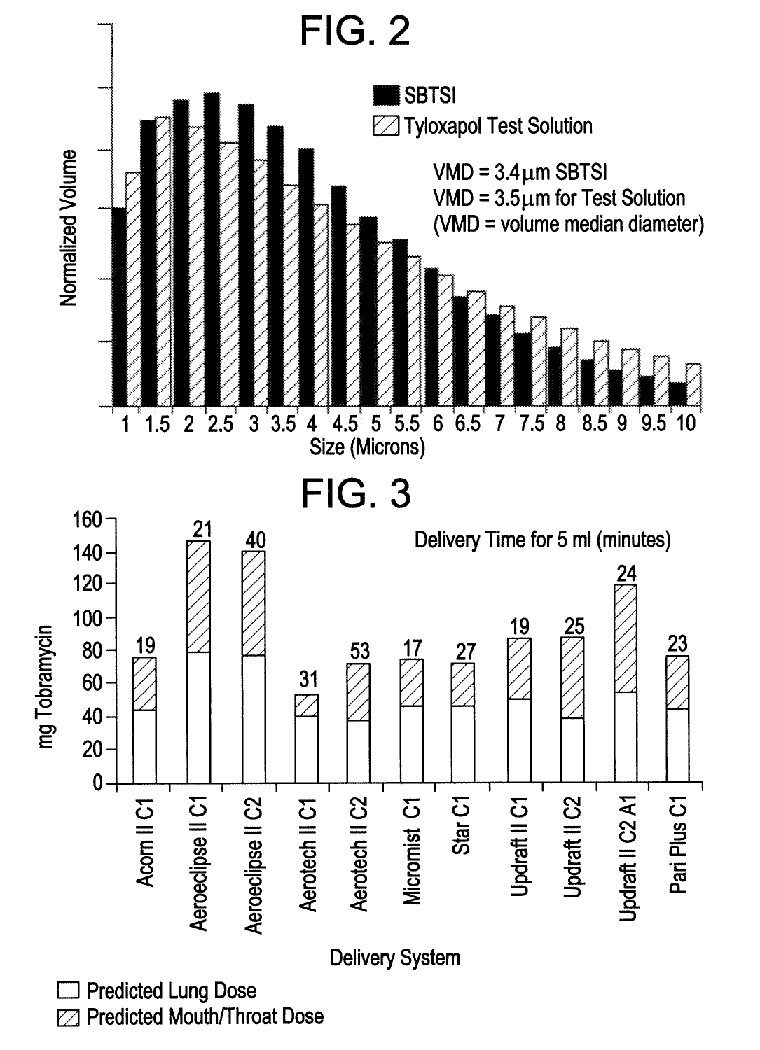

Before testing the various delivery systems, the inventors empirically verified that these two expediencies were acceptable (see FIGS. 1 and 2, infra). To verify that radioactivity associated with the Technetium DTPA radioisotope tag would accurately represent tobramycin drug mass, they nebulized SBTSI-Technetium DTPA solution with an AeroEclipse II nebulizer connected to an 8650D high-flow compressor. An Andersen cascade impactor (Thermo Scientific, Waltham, Mass.) physically separated aerosol particles into nine aerosol size ranges. Using mass spectrometry, the inventors measured tobramycin concentration in each aerosol size range. The results listed in FIG. 1 show that tobramycin concentration was proportional to the radioactive counts in each size range.

In order to verify that tagged tyloxapol is an accurate proxy for a surfactant-based antimicrobial solution for inhalation, the inventors used a Malvern Mastersizer S laser-diffraction instrument (Malvern Instruments, Ltd., Worcestershire, U.K.) to perform aerosol size comparisons on aerosolized SBTSI and aerosolized tyloxapol, each at identical concentrations. Both solutions were aerosolized by an AeroEclipse II nebulizer and an 8650D high-flow compressor. FIG. 2 shows the results verifying that the aerosol size for both solutions had similar median diameters and overall volume distributions.

After verifying that a tyloxapol-Technetium DTPA solution was an accurate proxy for SBTSI, the inventors tested each of the above-mentioned delivery systems. They gathered two important measurements from each delivery system: output rate and aerosol size. They then performed three output rate simulations and made thirty aerosol-size measurements for each of the eleven delivery systems. Output rate was measured by means of a breathing simulator (Harvard Lung, Harvard Apparatus, Holliston, Mass.) and high-efficiency filters (HEPA Lites, product of Teleflex Medical, Research Triangle Park, N.C.) that captured aerosolized particles emitted by the simulated inhalation. Using a Malvern instrument, the inventors also measured median aerosol size and relative aerosol volume in different size ranges. Corcoran et al., Am. J. Transplant 6: 2765-73 (2006), relates the methodology used to find output rate and aerosol size.

The inventors applied these output rate and aerosol size measurements to an in vitro model, which estimated the deposited dose received from a nebulizer treatment. The in vitro model was derived from past deposition studies of monosized aerosols and estimates total, pulmonary, and extrathoracic deposited dose. The inventors previously employed radioisotope aerosol dose quantification tests to quantify the accuracy of the in vitro model. Testing of the in vitro model verified that it could accurately estimate the dose of medication delivered by a nebulizer. See Corcoran et al. (2006), supra, for further discussion of the in vitro model.

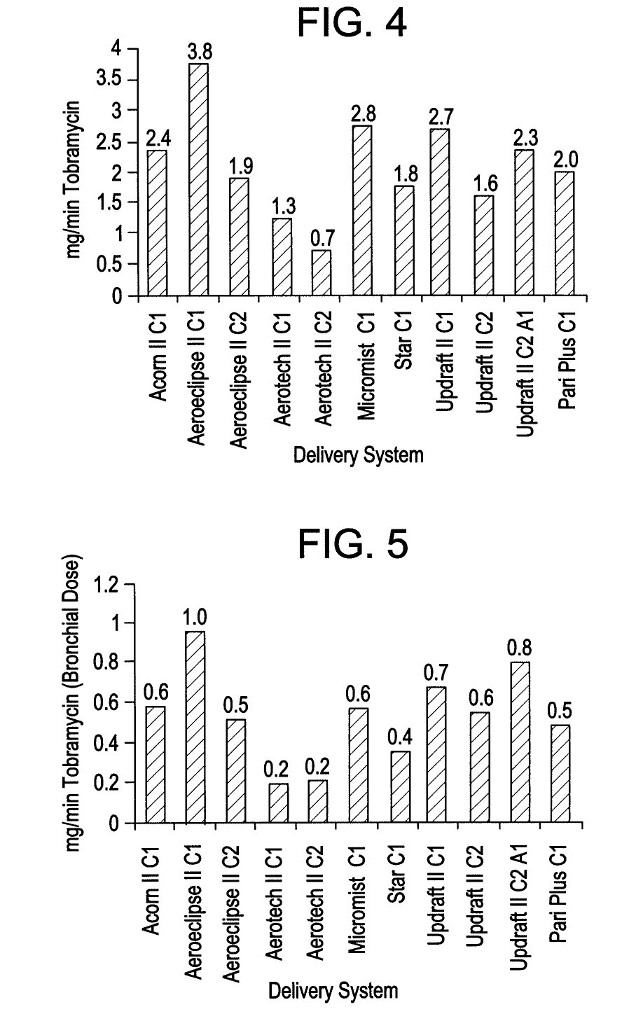

FIG. 3 displays the results after the inventors used the in vitro model to predict pulmonary and total deposited tobramycin doses for 5 ml of drug solution administered from each of the eleven delivery systems tested. Treatment time, measured during output rate testing (see FIG. 4), varied for the eleven delivery systems and should be taken into account when examining predicted doses. The AeroEclipse II Breath-Actuated Nebulizer combined with the DeVilbiss 8650D high-flow compressor (hereafter, “the second delivery system”) had the highest predicted total and pulmonary delivery doses.

FIG. 4 lists the results from the in vitro model for the eleven delivery systems in terms of predicted pulmonary delivery rate (predicted milligrams of tobramycin per minute). The second delivery system provided the greatest pulmonary dose delivery rate.

FIG. 5 lists the predicted bronchial delivery rate based on measurements from the in vitro model. This metric is especially useful because the primary sites of infection resulting from cystic fibrosis are the large and small airways of the lungs. The second delivery system yielded the highest bronchial dose delivery rate.

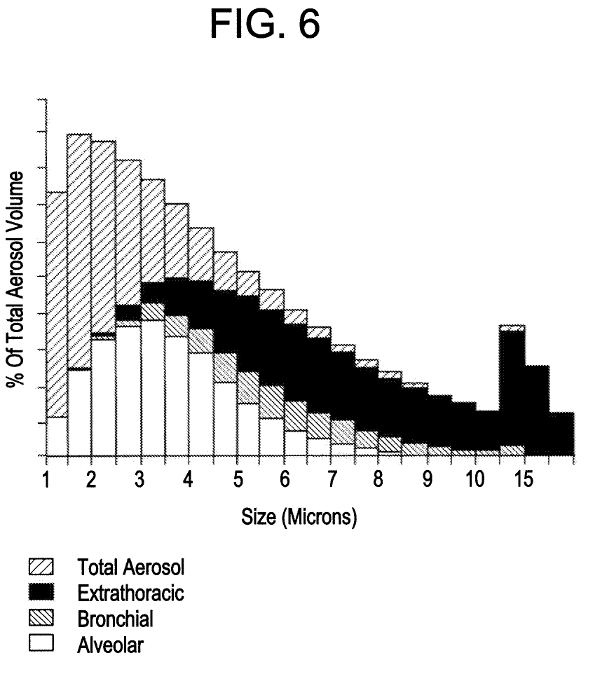

Based on the metrics described above, the second delivery system was identified as the optimal delivery system for a tyloxapol-based antimicrobial solution for inhalation. With this optimal system, the inventors determined the aerosol volume distribution by respiratory tract region (see FIG. 6). This histogram shows twenty-two particle size ranges. For each size range, the inventors predicted what percent of total aerosol volume comprised aerosol particles from the size range and what region of the respiratory tract on which particles from that size range were deposited.

### EXAMPLE 2

### In Vitro Methodology for Determining Dispersion Characteristics of Formulation of the Invention

A micropump nebulizer such as the Aerogen Pro, a product of Nektar/Aerogen (Sunnyvale, Calif.) is employed to produce a 4- to 5-micron median diameter aerosol, and tubing of decreasing diameter is used to deliver this aerosol through a 2 mm cannula tip. The aerosol is driven through the tubing system by means of a small air compressor, such as the Pulmoaide, a product of Sunrise Medical (Somerset, Pa.). The air is humidified and heated to 37° C. via a humidification system such as the MR850, a product of Fisher & Paykel Healthcare (Laguna Hills, Calif.). A flow meter placed upstream of the nebulizer is used to monitor and control air flow rate. The cannula tip is placed through a hole drilled in a cell culture plate lid that fixed its position 1 mm above the delivery surface.

Three primary delivery surfaces are used: (1) porcine gastric mucus (PGM), (2) human bronchial epithelial cell cultures from non-cystic fibrosis lungs (non-CF HBEs), and (3) human bronchial epithelial cell cultures from cystic fibrosis lungs (CF-HBEs). For (1), the PGM is mixed to a concentration of 50 mg/ml (95% saline) and loaded into 12 mm diameter filter inserts, e.g., the Corning-Costar Transwell Collagen T-cols (Acton, Mass.) to a depth of 4 mm. The cannula is placed about 1 mm above the PGM surface, and aerosol is delivered at 0.3 LPM for 10 seconds. For non-CF HBE and CF-HBE cases, epithelial cells are chemically detached from airway samples, cultured according to standard procedures, and seeded onto 12 mm transwell filters, where they are maintained at an air liquid interface until fully differentiated. Cultures are washed weekly, using applicable detergents, and also washed approximately 24 hours before testing. Immediately prior to aerosol delivery, the apical surface of the cells is hydrated with 100 μL of PBS solution, which then is suctioned off. Aerosol delivery is conducted onto the cells in a manner similar to that described for PGM surfaces.

Fluorescent probes are added to the solutions being tested, allowing for visualization of dispersion on the delivery surfaces. Three probes are utilized: a hydrophilic saccharide such as Texas red dextran (Molecular Probes, Carlsbad, Calif.); a polystyrene sphere of the order of 0.1 micron in diameter; and a polystyrene sphere of the order of 1 micron in diameter, such as FluoSpheres (Molecular Probes, Carlsbad, Calif.). Dispersion of the probes is tracked after delivery by means of a fluorescent dissecting microscope, e.g., the MVX10 MacroView, marketed by Olympus (Center Valley, Pa.). Dispersion of the test solutions is compared to an isotonic saline control that contains similar probes and delivered in a similar manner. Dispersion area is quantified using software tools such as MetaMorph, a product of Molecular Devices Corporation (Sunnyvale, Calif.). Approximately 8-fold increases in dispersion area vs. saline would be anticipated for a successful candidate solution on a PGM surface. Approximately 2-fold increases in dispersion area vs. saline would be anticipated for a successful candidate solution on both CF-HBE and non-CF HBE surfaces.

What is claimed is:

1. A sterile, isotonic aqueous composition comprised of an antimicrobial agent and a non-ionic surfactant, wherein the composition (a) does not comprise a phospholipid and (b) has a surface tension of about 35 dynes/cm or less.

2. A composition according to claim 1, wherein said antimicrobial agent is tobramycin and is present in an amount up to about 10% by mass.

3. A composition according to claim 1, wherein said non-ionic surfactant is tyloxapol that is present in an amount that is less than about 1% by mass.

4. A composition according to claim 3, wherein said tyloxapol is present in amount of about 0.1% by mass.

5. A composition according to claim 1, wherein said composition is aerosolized.

6. A composition according to claim 1, wherein said antimicrobial agent is an antibiotic agent, an antifungal agent, an antiviral agent, or a combination thereof.

7. A composition according to claim 6, wherein said antimicrobial agent is at least one of tobramycin, amakacin, ceftazidime, aztreonam, colistin, ciprofloxacin, azithromycin, pentamidine, and gentamicin.

8. A composition according to claim 6, wherein said antimicrobial agent is at least one of vancomycin, doxycycline, linezolid, meropenem, and tigecycline.

9. A composition according to claim 6, wherein said antimicrobial agent is at least one of isoniazid, rifampin, and daptomycin.

10. A composition according to claim 6, wherein said antimicrobial agent is Amphotericin B.

11. A composition according to claim 6, wherein said antimicrobial agent is at least one of zanamivir and oseltamivir.

12. A composition according to claim 6, wherein said surface tension is about 35 dynes/cm.

13. A composition according to claim 6, wherein said surface tension is less than about 35 dynes/cm.

14. A combination for delivering an antimicrobial agent to the lungs by oral inhalation, comprising (A) a breath-actuated nebulizer operatively connected to (B) a high-flow compressor that delivers to said nebulizer a gas flow greater than 5 L/min and a pressure head of at least 40 psi, wherein said nebulizer contains a liquid to be atomized, which liquid is an aqueous composition according to claim 1.

15. A method for delivering an antimicrobial agent to the lungs, comprising (A) forming an aerosol of an aqueous composition according to claim 1, wherein said aerosol is characterized by a median droplet size in the range of about 1 to 5 μm, and (B) delivering said aerosol to a subject for inhalation, such that said subject receives aerosol only during inhalation.

16. A composition according to claim 2, wherein said composition is aerosolized.

17. A composition according to claim 3, wherein said composition is aerosolized.

18. A composition according to claim 4, wherein said composition is aerosolized.
